(19) United States
(12) Patent Application Publication
(10) Pub. No.: US 2020/0353175 A1
(43) Pub. Date: Nov. 12, 2020

(54) Injection Device with a Preselector

(71) Applicant: Sanofi-Aventis Deutschland GMBH, Frankfurt am Main (DE)

(72) Inventors: Michael Helmer, Frankfurt am Main (DE); Ernst Raab, Frankfurt am Main (DE)

(21) Appl. No.: 16/761,110

(22) PCT Filed: Nov. 5, 2018
(86) PCT No.: PCT/EP2018/080080

§ 371 Date: May 1, 2020

(30) Foreign Application Priority Data
Nov. 7, 2017 (EP) 17200316.2

(51) Int. Cl. A61M 5/315

(52) U.S. Cl. CPC A61M 5/31536; A61M 5/3155; A61M 2005/3125; A61M 2205/583; A61M 2205/0272; A61M 2205/582

(57) ABSTRACT

An injection device for setting and injecting a dose of a medicament is provided. The injection device includes: an elongated housing extending along a longitudinal axis (z) and comprising a sidewall, a dose tracker having a portion arranged inside the housing, the dose tracker comprising at least one tracking stop feature, wherein a positional state of the dose tracker relative to the housing is indicative of a size of the dose, and a preselector comprising a preselector stop feature. Wherein the tracking stop feature and the preselector stop feature are configured to mutually engage and to block a displacement of the dose tracker beyond a predefined maximum dose positional or rotational state. The preselector is at least one of translationally or rotationally displaceable relative to the housing along a displacement path between at least two preselection positional states.

### CROSS REFERENCE TO RELATED APPLICATIONS

The present application is the national stage entry of International Patent Application No. PCT/EP2018/080080, filed on Nov. 5, 2018, and claims priority to Application No. EP 17200316.2, filed on Nov. 7, 2017, the disclosures of which are incorporated herein by reference.

### TECHNICAL FIELD

The present disclosure relates in one aspect to injection devices, such as a pen-type injector for setting and dispensing of a dose of a medicament. The disclosure relates to an injection device providing a maximum dose mechanism, i.e. a dose setting and dispensing mechanism that is only operable to dispense a dose that does not exceed a predefined maximum threshold.

### BACKGROUND

Injection devices for setting and dispensing a single or multiple doses of a liquid medicament are as such well-known in the art. Generally, such devices have substantially a similar purpose as that of an ordinary syringe.

Injection devices, in particular pen-type injectors have to meet a number of user-specific requirements. For instance, with patient's suffering chronic diseases, such as diabetes, the patient may be physically infirm and may also have impaired vision. Suitable injection devices especially intended for home medication therefore need to be robust in construction and should be easy to use. Furthermore, manipulation and general handling of the device and its components should be intelligible and easy understandable. Moreover, the dose setting as well as dose dispensing procedure must be easy to operate and has to be unambiguous.

Typically, such devices comprise a housing including a particular cartridge holder, adapted to receive a cartridge at least partially filled with the medicament to be dispensed. Such devices further comprise a drive mechanism, usually having a displaceable piston rod which is adapted to operably engage with a piston of the cartridge. By means of the drive mechanism and its piston rod, the piston of the cartridge is displaceable in a distal direction or dispensing direction and may therefore expel a predefined amount of the medicament via a piercing assembly, which is to be releasably coupled with a distal end section of the housing of the injection device.

The medicament to be dispensed by the injection device is provided and contained in a multi-dose cartridge. Such cartridges typically comprise a vitreous barrel sealed in a distal direction by means of a pierceable seal and being further sealed in proximal direction by the piston. With reusable injection devices an empty cartridge is replaceable by a new one. In contrast, injection devices of disposable type are to be discarded when the medicament in the cartridge has been dispensed or used-up.

### SUMMARY

For some applications it can be advantageous to limit a maximum size of a dose that can be dispensed or expelled from the cartridge. Then, unintended overdosing of the medicament could be prevented.

The present disclosure provides an injection device that provides a maximum dose function or a maximum dose preselector. The maximum dose function or the maximum dose preselector should be easily adaptable to existing designs of injection devices. The maximum dose function or maximum dose preselector should be also read configurable on demand and should provide an easy and intuitive approach to vary the size of the maximum dose that can be expelled with the injection device. It is a further aim to provide an injection device with a limited capability to set and to dispense doses of different sizes. In particular the injection device should be configured to allow and enable setting and dispensing of only a few, e.g. of 2, 3 or 4 differently sized doses of the medicament.

An implementation of a maximum dose function should be achievable by only modifying a limited number of existing device components. It is a further aim to individually modify maximum dose values or dose sizes by changing only a single component or just a few components of the device. Hence, the maximum dose function of the device or its drive mechanism should be configurable by changing only one or a few components of the device or of its dose setting mechanism or drive mechanism. It is a particular aim to transfer an injection device configured for individual selection and dispensing of differently sized doses towards a fixed dose injection device providing a limited or restricted dose size selection.

The maximum dose function or maximum dose preselector should be universally applicable to a large variety of drive mechanisms and injection devices. In particular, the maximum dose function or maximum dose preselector should be equally applicable to disposable injection devices as well as to reusable injection devices.

In one aspect there is provided an injection device for setting and dispensing, hence injecting of a dose of a medicament. The injection device comprises an elongated housing extending along a longitudinal axis. The housing is configured and sized to accommodate a dose setting mechanism. Typically, the housing is also sized and configured to accommodate a cartridge filled with a medicament. The housing comprises a side wall. Typically, the housing is of cylindrical or tubular shape. A cylinder long axis extends in axial direction that coincides or extends parallel to the longitudinal axis of the housing and the sidewall is of tubular or cylindrical shape. The geometric shape of the sidewall of the housing may therefore define a radial direction and a circumferential direction.

The injection device and/or the dose setting mechanism thereof further comprises a dose tracker having a portion that is arranged inside the housing. The dose tracker may be partially arranged inside the housing. It may be entirely arranged inside the housing or it may be configured to at least partially protrude from the housing, e.g. in longitudinal direction from a proximal end of the housing. The dose tracker can be operably connectable to a dose dial. The dose dial may be configured to set or to dial a dose of predefined size. For this the dose dial may be operably connectable to the dose tracker. Alternatively, the dose tracker itself may be directly actuatable by a user for setting of a dose.

The dose tracker comprises at least one tracking stop feature. The dose tracker is at least one of translationally or rotationally displaceable relative to the housing for and during setting of a dose. A positional state of the dose tracker relative to the housing is indicative of a size of the dose. In the present context a ‘positional state’ of a component, e.g. of the dose tracker includes a position of the component and an angular orientation of the component relative to another component, e.g. relative to the housing.

The dose tracker may belong to the dose setting mechanism. The positional state of the dose tracker relative to the housing is unequivocally correlated to the size of the dose actually set. Depending on the specific implementation of the dose tracker a degree of rotation and/or a degree of longitudinal or axial translation of the dose tracker relative to the housing is indicative of a size of the dose actually set.

The injection device further comprises a preselector comprising a preselector stop feature. The preselector stop feature and the tracking stop feature are configured to mutually engage and to block a displacement of the dose tracker beyond a predefined maximum dose positional state or beyond a predefined maximum dose rotational state.

The preselector is displaceable relative to the housing along the longitudinal axis. The preselector is lockable to the housing, in particular to the sidewall thereof in at least two different positional states relative to the housing. Here, the positional state may refer to a longitudinal position of the preselector relative to the housing. The positional state may also refer to an angular orientation of the preselector relative to the housing when the preselector is rotatably supported in or relative to the housing.

Typically, the preselector is displaceable at least between a first preselection positional state and a second preselection positional state. The first preselection positional state may coincide with a distal stop position of preselector relative to the housing. The second preselection positional state may coincide with a proximal stop position of the preselector relative to the housing. In the second preselection positional state the preselector may be located closer to a proximal end of the housing in the first preselection positional state. In any of the at least two preselection positional states the preselector is lockable to the housing.

A specific portion of the sidewall of the housing, to which the preselector is lockable defines a positional state at which a longitudinal or rotational displacement of the tracking stop feature is blocked. Hence, the tracking stop feature and the dose tracker are hindered from moving beyond the positional or rotational state defined by the position of the preselector relative to the sidewall of the housing.

In other words, when the dose tracker reaches a maximum dose positional state its tracking stop feature engages, e.g. abuts with the preselector stop feature that is at least temporally locked to a predefined portion or section of the sidewall. The predefined portion or section of the sidewall may be defined by a specific position with regard to the longitudinal axis and/or by a specific position with regard to a tangential or circumferential direction of the sidewall. The preselector stop feature and the tracking stop feature may comprise mutually corresponding stop faces, e.g. extending in circumferential and/or radial direction so as to engage axially. Alternatively or additionally, the preselector stop feature and the tracking stop feature comprise mutually corresponding stop faces extending in axial direction and radial direction so as to engage circumferentially. When configured to engage axially, the mutual engagement of the preselector stop feature and the tracking stop feature provides an axial stop thereby impeding and blocking a longitudinal or axial translation of the dose tracker beyond a predefined maximum axial dose positional state.

When configured to engage circumferentially or tangentially, the mutual engagement of the preselector stop feature and the tracking stop feature provides a rotational stop, thereby impeding and blocking a rotation of the dose tracker relative to the housing beyond a predefined maximum rotational dose positional state.

The predefined maximum dose positional state defines a maximum dose that is selectable and dispensable by the injection device. By placing and locking the preselector stop feature to a predefined location on the sidewall of the housing that corresponds to the positional state of the dose tracker, in particular of the tracking stop feature, the maximum dose dispensable by the injection device can be defined.

In some examples the injection device and/or the dose setting mechanism thereof comprises a dose dial. The dose dial is rotatable or translationally displaceable relative to the housing for setting of a dose. The dose dial may be rotatably supported on or in the housing. For instance, the dose dial may be rotatably supported at a proximal end section of the housing. The dose dial is user actuatable. Hence, a user may grip and rotate the dose dial relative to the housing for setting or selecting of a dose of variable size. The dose dial may comprise or may form a dose dial. The dose dial may comprise a rotatable knob or ring, e.g. provided at a proximal end of the housing of the injection device.

According to one example the preselector is slidably displaceable along a displacement path extending along the sidewall of the housing. The preselector is slidingly engaged with the sidewall of the housing. It is longitudinally and/or axially guided by the housing when displaced along the displacement path. The displacement path extends parallel to the longitudinal axis. It is linearly or straight shaped. The displacement path has a proximal end and a distal end. When the preselector is in the distal stop position it is at or close to the distal end of the displacement path. When the preselector is in the proximal stop position it is located at or close to the proximal end of the displacement path. Typically, the displacement path is visible or discernible from outside the injection device.

The preselector may be accessible from outside the injection device. It may be configured for manual displacement by a user between the proximal stop position, the distal stop position or any longitudinal or axial position located therebetween.

A sliding displacement of the preselector relative and along the housing provides a rather intuitive approach to modify the axial position of the preselector in order to modify the maximum dose size of the dose setting mechanism of the injection device.

According to another example the preselector is rotationally locked to the housing. In this way the preselector is exclusively longitudinally displaceable relative to the housing. At least a portion of the preselector, e.g. a sleeve portion thereof is located inside the housing. An outside facing surface of the preselector, in particular an outside facing surface of the sleeve portion of the preselector may comprise a spline feature to engage with a correspondingly shaped spline feature of the housing. At least one of the spline features of the preselector and the housing comprises a radial protrusion and the other one of the spline features of the preselector and the housing comprises a correspondingly shaped recess.

The protrusion is a radial protrusion and the recess is a radial recess. At least one of the radial recess and the radial protrusion extends along the longitudinal axis thus allowing a sliding displacement of the preselector relative to the housing, hence relative to the sidewall of the housing. Typically, the preselector comprises a longitudinally elongated rib protruding radially from an outside surface of a sleeve portion of the preselector. The rib is axially guided in a correspondingly shaped recess at an inside facing portion of the sidewall of the housing.

In a further example the preselector comprises a sleeve portion enclosing a longitudinal portion of the dose tracker. The dose tracker may comprise a number sleeve or may form a number sleeve having an outside facing surface printed or provided with dose indicating numbers or other dose size indicating symbols or characters. In other embodiments the dose tracker comprises a tubular or sleeve like shape but is void of dose indicating numbers on an outside surface thereof. With the preselector comprising a sleeve portion the preselector may circumferentially enclose the tubular shaped dose tracker.

The dose tracker may comprise a helical thread or a helically shaped protrusion on an outside surface to engage with a correspondingly shaped helical structure on an inside surface of the sidewall of the housing. In other words, the dose tracker, hence the number sleeve and the sidewall of the housing may be threadedly engaged. A rotation of the dose tracker, e.g. during and/or for setting of a dose also transfers into a longitudinal displacement of the dose tracker relative to the housing. Typically, during setting of a dose the preselector is positionally or rotationally locked to the sidewall of the housing. During setting of a dose the preselector remains stationary relative to the housing. In this way it defines the maximum dose size and limits a maximum displacement of the dose tracker starting from a zero dose configuration or zero dose position towards a maximum dose configuration or maximum dose position.

With the sleeve portion at least enclosing a section of the dose tracker a rather stable abutment configuration or blocking configuration between the tracking stop feature and the preselector stop feature can be obtained. Due to a circumferential or partially circumferential enclosure of the dose tracker by the sleeve portion of the preselector the dose tracker can be axially and radially guided by the sleeve portion of the preselector.

In another example the preselector comprises a slider slidably arranged in a recess in an outside facing portion of the sidewall. The recess is provided as a recessed structure in the outside surface of the sidewall of the housing. The recess may comprise a bottom as well as oppositely located sidewalls, e.g. extending along the longitudinal axis. The bottom and the sidewalls of the recess may provide sliding surfaces or sliding structures along which the slider is slidably guided. The bottom of the recess typically comprises at least a longitudinal slit through which the slider may extend or through which the slider is connected to the sleeve portion of the preselector.

When the recess of the sidewall of the housing comprises a bottom the preselector may comprise two separate pieces, namely the sleeve portion assembled inside the housing and the slider assembled to the outside of the housing. The slider and the sleeve portion may be mutually connected through the slit provided in the bottom of the recess.

In other examples the recess of the sidewall of the housing may comprise a through opening entirely covered by the slider. Here, the slider may be located inside the housing and may be accessible through the recess from outside the housing for displacing the slider between the proximal stop position, the distal stop position and optional intermediate positions therebetween.

Here, the slider and the sleeve portion of the preselector may be integrally formed. They may represent portions of a single pieced preselector. A single piece preselector may be easy to manufacture and to assemble. However, insertion of a single piece preselector into the interior of the housing when the preselector comprises a sleeve portion and a slider integrally formed therewith may cause some difficulties, especially in a fully automated assembly process. Having a preselector with two separate and mutually connectable components, such as the sleeve portion and the slider, may be beneficial for the assembly process. Here, the sleeve portion may be arranged inside the housing while after insertion of the sleeve portion into the housing the slider is arranged in the recess of the sidewall and is hence connected, e.g. clipped to the slider.

Here, the slider and the sleeve portion comprise mutually corresponding connectors, which, when engaged, extend through a slit in the bottom of the recess.

According to another example the slider comprises a detent structure configured to engage with a correspondingly shaped counter detent structure of the sidewall. By means of the detent structure and the counter detent structure the slider can be fixed and locked to the housing, hence to the sidewall thereof, in at least two different longitudinal or circumferential positions relative to the housing. If the preselector is displaceable relative to the longitudinal axis the recess is of elongated structure and extends along the longitudinal axis of the housing. In configurations where the preselector is displaceable relative to the housing along a tangential or circumferential direction also the recess extends along the tangential or circumferential direction of the sidewall of the housing.

By means of the detent structure the slider is lockable to the housing in selected and predefined longitudinal or tangential positions that define a maximum dose configuration, hence a maximum dose position or maximum dose orientation of the dose tracker.

The detent structure and the counter detent structure enable a well-defined positional interlocking of the preselector relative to the sidewall of the housing. The mechanical engagement of the detent structure and the counter detent structure provides a well-defined holding force. A user intending to displace the preselector relative to the housing must exert a displacement force being larger than the holding force between the detent structure and the counter detent structure. The detent structure and the counter detent structure provide or form a kind of a ratchet engagement by way of which the preselector can be fixed and locked to the housing in a number of discrete longitudinal or tangential positions relative to the housing.

According to another example one of the detent structure and the counter detent structure comprises a protrusion and the other one of the detent structure and the counter detent structure comprises at least two recesses. Any one of the at least two recesses is configured and/or shaped to receive the protrusion in order to lock or to fix the preselector against movement relative to the housing.

It is conceivable that the protrusion is a radial protrusion and that the at least two recesses are radial recesses that are separated along the displacement path, e.g. along the longitudinal axis and/or along the tangential or circumferential direction of the housing. The protrusion engages with another of the two recesses when the preselector is in a different preselection positional state, e.g. a specific longitudinal position relative to the housing. When the displacement path extends along a parallel to the longitudinal direction the protrusion and the correspondingly shaped recesses extend in tangential direction. The recesses are separated in longitudinal direction.

In another example it is also conceivable, that the protrusion is an axial protrusion and that the at least two recesses are configured as axial recesses. Here, the axial recesses are separated along the tangential or circumferential direction of the housing. In this way, the protrusion can be locked and fixed in a first of the two recesses when the preselector is in a first rotational state relative to the housing. The protrusion can be fixed in a second recess when the preselector is in a second rotational state relative to the housing.

The mutually engaging detent structure and counter detent structure also provides an audible as well as a haptic feedback to the user when the slider is displaced either longitudinally or tangentially relative to the housing. Insofar, the mutually engaging detent structure and counter detent structure provides a twofold function. First of all the mutually engaging detent structures define discrete longitudinal or tangential positions of the preselector and/or of the slider. Second, the mutually engaging detent structure and the counter detent structure provide haptic and/or audible feedback when the preselector is manually displaced by a user in order to modify the maximum dose size of the dose setting mechanism.

Typically, the counter detent structure is located close to or in the recess of the sidewall of the housing. The counter detent structure may be integrated into the recess. It may be provided in a sidewall of the recess or may extend along a side edge of the recess.

In another example the preselector comprises a radially outwardly facing gripping structure comprising at least two radially outwardly protruding ribs that are separated from each other along the displacement path. Depending on orientation or extension of the displacement path the protruding ribs are separated from each other along the longitudinal axis or along the tangential direction. With a slider displaceable in longitudinal direction the at least two radially outwardly protruding ribs are separated from each other along the longitudinal axis. With the preselector slidably displaceable relative to the housing along the tangential direction the at least two radially outwardly protruding ribs are separated in tangential direction.

The radially outwardly protruding ribs may provide or form a rippled structure thus enabling an easy gripping and/or an effective non-slipping engagement between a finger of a user and the preselector. Typically, the gripping structure is provided on an outside surface of the slider of the preselector. The gripping structure or its radially outwardly protruding ribs may also contribute to the detent structure of the slider. Insofar the gripping structure may provide a twofold function. On the one hand it may enhance and improve a sliding engagement between a finger of a user, a tool used by the user and the slider. On the other hand the gripping structure and/or at least one of the radially outwardly protruding ribs may engage with the counter detent structure of the sidewall of the housing.

According to a further example the gripping structure is radially recessed from an outside surface of the sidewall, the gripping structure substantially flushes with the outside surface of the sidewall or the gripping structure protrudes radially outwardly from the outside surface of the sidewall. In the recessed or flush configuration the gripping structure is not protruding from the outside surface of the sidewall. In this way, an unintentional displacement of the gripping structure and hence of the slider relative to the housing can be effectively prevented. The recessed or a flush mounted arrangement of the gripping structure relative to the outside surface of the sidewall of the housing may require a dedicated and intentional manipulation of the gripping structure and hence of the slider in order to move the preselector from one discrete position to another discrete position relative to the housing.

As with the recessed or flush mounted arrangement of the gripping structure it is conceivable, that the distance between at least two of the radially outwardly protruding ribs of the gripping structure is selected or designed in accordance with the size of a dedicated displacement tool.

The displacement tool may comprise a protrusion that fits into the space between the at least two radially outwardly protruding ribs of the gripping structure. The dedicated tool may comprise a screwdriver. It is also conceivable, that a portion of the injection device serves as the dedicated tool. For instance, a protrusion of a protective cap intended to cover the distal dispensing end of the injection device may be configured to fit into the space between the protruding ribs of the gripping structure. In this way, the protective cap may serve and provide a dedicated tool for displacing the preselector. Otherwise the preselector may be hardly reachable or displaceable when located recessed or flush mounted with regard to the outside surface of the housing.

In other configurations in which the gripping structure protrudes radially outwardly from the outside surface of the sidewall the gripping structure and hence the slider provided with the gripping structure is directly displaceable, e.g. by a finger of a user's hand. Here, the radially outwardly protruding ribs may provide a rippled structure providing a slip-free engagement between a user's finger and the slider.

In another example the preselector comprises at least one preselection indication on an outside facing surface portion. Furthermore, the housing comprises a preselection window to reveal the preselection indication of the preselector or the housing accommodates or comprises a position sensor configured to determine the position of the preselection indication, e.g. along the longitudinal axis or with regard to a tangential direction.

The preselection indication may comprise a number, a symbol, a texture or a color. The preselection indication may be provided on an outside facing portion of the sleeve portion of the preselector. Since the sleeve portion is located inside the housing the preselection indication may reveal or may show up in a preselection window provided in the housing of the injection device. The preselection window may be provided as a second window of the housing. The housing may further comprise a first or main window, also denoted as dosage window in which numbers of a dose scale of the dose setting mechanism may show up in addition to the preselection indication.

The preselection indication may comprise numerous numbers or symbols, such as numbers 1, 2, 3. Here, each number may represent a multiple of units of the medicament to be dispensed. The preselection indication is indicative of the maximum settable dose side of the dose setting mechanism. For instance, a number 1 of a preselection indication may represent 10 international units of a medicament, a preselection indication with number 2 may represent 20 international units and a preselection indication number 3 may represent 30 international units. Depending on the position of the preselector a respective preselection indication will show up in the preselection window. If for instance a preselection indication number 2 shows up in a preselection window the user is informed, that a maximum dose size of 20 international units can be set. The dose size actually set and dialed during a dose setting procedure may show up in the dosage window of the housing.

Alternative to the housing comprising a preselection window or in addition to such a housing the housing may also accommodate or may comprise a position sensor that is configured to determine the position of the preselection indication. The position sensor may also belong to the injection device or it may belong to an auxiliary or add-on device attachable to the injection device. The position sensor is configured to determine the longitudinal or tangential position of the at least one preselection indication. The position sensor may be implemented as an electronic position sensor. In this way, the specific position, i.e. the longitudinal position or tangential or circumferential position of the preselector or of its slider can be determined electronically. An electronically determined position of the preselector can be further processed by an auxiliary device or add-on device, e.g. for recording or acquisition of a dosing and/or of a dispensing history of the injection device.

In a further example the housing comprises at least two preselection indications arranged along the displacement path of the preselector. Here, the preselection indications, e.g. numbers 1, 2, 3 or other symbols may be provided on the outside surface of the housing. The at least two preselection indications may be separated along the longitudinal extension of the displacement path. With the preselector displaceable along the displacement path, e.g. along the longitudinal axis of the housing also the preselection indications are separated along the longitudinal axis. With a tangential or circumferential sliding displacement of the preselector the preselection indications are separated along the tangential or circumferential direction, respectively. While the at least two preselection indications are arranged along the displacement path, typically at the outside surface of the sidewall of the housing the preselector comprises a pointer coinciding with the position of at least one of the at least two preselection indications. With a longitudinally displaceable preselector the pointer longitudinally coincides with the position of at least one of the at least two preselection indications. With a tangentially or circumferentially displaceable preselector the pointer aligns or coincides axially with the position of at least one of the at least two preselection indications.

In this way, the pointer of the preselector is directly indicative of the pre-selected maximum dose size which is determined by the position of the preselector relative to the housing. In any of the discrete positional stages of the preselector relative to the housing the pointer points to only one of the at least two preselection indications. The pointer is hence indicative of the preselection indication that corresponds to the momentary positional state of the preselector relative to the housing.

The pointer is typically provided on an outside facing surface of the slider of the preselector. In this way and since the slider is typically discernible from outside the device also the pointer and its alignment with a respective preselection indication is directly visible to a user of the device.

In another example an outside surface of the dose tracker comprises a first surface section and a second surface section. At least one of a haptic appearance, a visual appearance, a magnetic property or an electric property of the second surface section differs from a respective haptic appearance, a visual appearance, a magnetic property or an electric property of the first surface section. In other words, the first surface section and the second surface section mutually distinguish with regard to their haptic appearance, with regard to their visual appearance, with regard to their magnetic properties or with regard to their electric properties. In this way the dose tracker can be either haptically, visually, magnetically and/or electrically encoded. Having non-overlapping first and second surface section with difference haptic, visual, magnetic or electric properties allows to provide an additional indication to a user if and in how far the dose tracker has reached a maximum dose positional state or a maximum dose configuration.

According to another example the preselector, e.g. the slider thereof, comprises an aperture radially extending through the preselector to reveal a portion of the outside surface of the dose tracker. Here it is particularly intended, that the second surface section of the dose tracker only reveals and coincides with the aperture of the preselector slider when the preselector stop feature engages the tracking stop feature. In all other configurations, wherein the preselector stop feature is separated from the tracking stop feature the first surface section of the dose tracker reveals or appears in the aperture of the preselector or slider.

In this way the first and second surface sections of the dose tracker in combination with the aperture of the slider provide an intuitive feedback to the user whether a maximum dose configuration has already been reached or not. As long as the first surface section appears in the aperture the user will be aware that the dose setting or dose dialing procedure has to be continued. It is only when the second surface section of the outside surface of the dose tracker appears in the aperture of the preselector or slider that the tracking stop feature engages with the preselector stop feature so as to block any further dose increasing displacement of the dose dial and hence of the dose tracker relative to the housing and hence relative to the preselector that is constrained to the housing. When reaching the mutual blocking configuration of the tracking stop feature and the preselector stop feature the second surface section will appear in the aperture thus providing a confirmation for the end user, that a maximum dose configuration has been reached and that a dose dispensing procedure may commence.

According to another example the slider comprises a magnifying lens arranged in the aperture. By means of the magnifying lens, the appearance of the surface section of the dose tracker that is visible through the aperture and hence through the lens can be enlarged. This allows for a minimization the diameter or cross-section of the aperture.

Generally, the preselector may be fixed in the preselection positional states at discrete positions relative to the housing or relative to the dose tracker. The supported preselection states may correspond to consecutive and complete revolutions of the dose tracker. Alternatively or additionally it is also conceivable that the dose tracker comprises two or even three tracking stop features to engage with the preselector stop feature. Alternatively, also the preselector may comprise two or more preselector stop features to engage with the tracking stop feature. In this way the maximum dose positional state could be assigned with every half or every third revolution of the dose tracker relative to the housing. Furthermore it is conceivable, that two or more tracking stop features simultaneously engage with correspondingly shaped two or more preselector stop features. In this way the mechanical interaction and robustness of the abutment between the dose tracker and the preselector can be enhanced and increased.

In another example the injection device further comprises a piston rod. The piston rod is typically a component of a drive mechanism and/or of the dose setting mechanism. The piston rod is axially displaceable for dispensing of a dose of a medicament from a cartridge. When the injection device is in a dispensing mode, the drive mechanism and/or the dose setting mechanism thereof is configured to drive and to displace the piston rod longitudinally in an axial distal direction.

Typically, the injection device is provided with a cartridge filled with a medicament, e.g. a liquid medicament. The cartridge is typically sealed in proximal direction by means of a bung. The bung is axially displaceable inside the cartridge so as to expel the liquid medicament from a distal end thereof. The distal end of the cartridge is typically sealed by a pierceable seal. The pierceable seal is penetrable by means of a double-tipped injection needle. The injection needle is typically releasably attachable to a distal and or to a dispensing end of the housing of the injection device, typically to a distal end of a cartridge holder that belongs to the housing of the injection device.

In a further example the injection device comprises a cartridge at least partially filled with a medicament. The cartridge comprises a barrel filled with the medicament. The cartridge and hence the barrel is sealed in an axial proximal direction by the bung. The bung is axially displaceable relative to the barrel by means of the piston rod. When during dose dispensing the piston rod advances in distal direction it applies a driving pressure to the bung. Since the cartridge is fixed inside the housing the bung starts to move in distal direction, thereby increasing an inside pressure of the cartridge, thus leading to expelling the medicament from the cartridge.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound, wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound, wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis, wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two μ sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains p and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by A and K. A light chain has two successive domains: one constant domain (CL) and one variable domain (VL). The approximate length of a light chain is 211 to 217 amino acids. Each antibody contains two light chains that are always identical; only one type of light chain, K or A, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present injection device without departing from the spirit and scope of what is disclosed herein. Further, it is to be noted, that any reference numerals used in the appended claims are not to be construed as limiting the scope of the invention.

### BRIEF DESCRIPTION OF THE FIGURES

In the following, example embodiments of the drive mechanism and the injection device are described in detail by making reference to the drawings, in which:

FIG. 1 schematically shows an example of an injection device;

FIG. 2 shows an exploded view of the components of the injection device of FIG. 1;

FIG. 3 shows one embodiment of a proximal end of an injection device with a preselector;

FIG. 4 shows a perspective view of the interior of the device according to FIG. 3;

FIG. 5 is a rotated view of the components of the device according to FIG. 4 without the housing;

FIG. 6 is a side view of the injection device after setting of a dose to a maximum dose size;

FIG. 7 shows a dedicated tool in form of the protective cap to displace the preselector;

FIG. 8 is a longitudinal cross-section through the device according to FIGS. 3-7;

FIG. 9 shows another implementation of a preselector in an injection device;

FIG. 10 is a perspective illustration of the interior of the device according to FIG. 9;

FIG. 11 shows a schematic side view of the preselector according to FIGS. 9 and 10;

FIG. 12 is a perspective view of an injection device comprising another implementation of a preselector;

FIG. 13 is another perspective view of the device according to FIG. 12;

FIG. 14 is an isolated view of components of the device according to FIGS. 12 and 13;

FIG. 15 is an isolated perspective illustration of the preselector of the device according to FIGS. 12-14;

FIG. 16 is a longitudinal cut through the device according to FIGS. 12-14;

FIG. 17 is a side view of the device according to FIGS. 12-14 with the preselector in a distal stop position;

FIG. 18 is a side view of the injection device with the preselector in an intermediate position;

FIG. 19 is a side view with the preselector in a proximal stop position;

FIG. 20 is a perspective isolated view of the dose tracker;

FIG. 21 shows a further embodiment of an injection device with an electronic display and with a preselector in an initial configuration with the preselector in a distal stop position;

FIG. 22 shows the device according to FIG. 21 with the dose dial and the dose tracker in a maximum dose configuration;

FIG. 23 shows the device according to FIGS. 21 and 22 with the preselector in a proximal stop position and with the dose dial and the dose tracker in a zero dose configuration;

FIG. 24 shows the device according to FIG. 23 with the dose dial and the dose tracker in the maximum dose configuration;

FIG. 25 shows a perspective view of the interior of the injection device according to FIGS. 21-24;

FIG. 26 shows an add-on device for attachment to a proximal end of the injection device according to FIGS. 21-25;

FIG. 27 is an exploded view of the add-on device detached from the housing of the injection device;

FIG. 28 is a bottom view of the add-on device;

FIG. 29 schematically shows the interaction of the add-on device with the dose tracker,

FIG. 30 is indicative of the dose tracker in a zero dose configuration;

FIG. 31 is indicative of the dose tracker in a first maximum dose configuration;

FIG. 32 is indicative of the dose tracker in a second maximum dose position or configuration; and

FIG. 33 is indicative of the dose tracker in a third maximum dose configuration.

### DETAILED DESCRIPTION

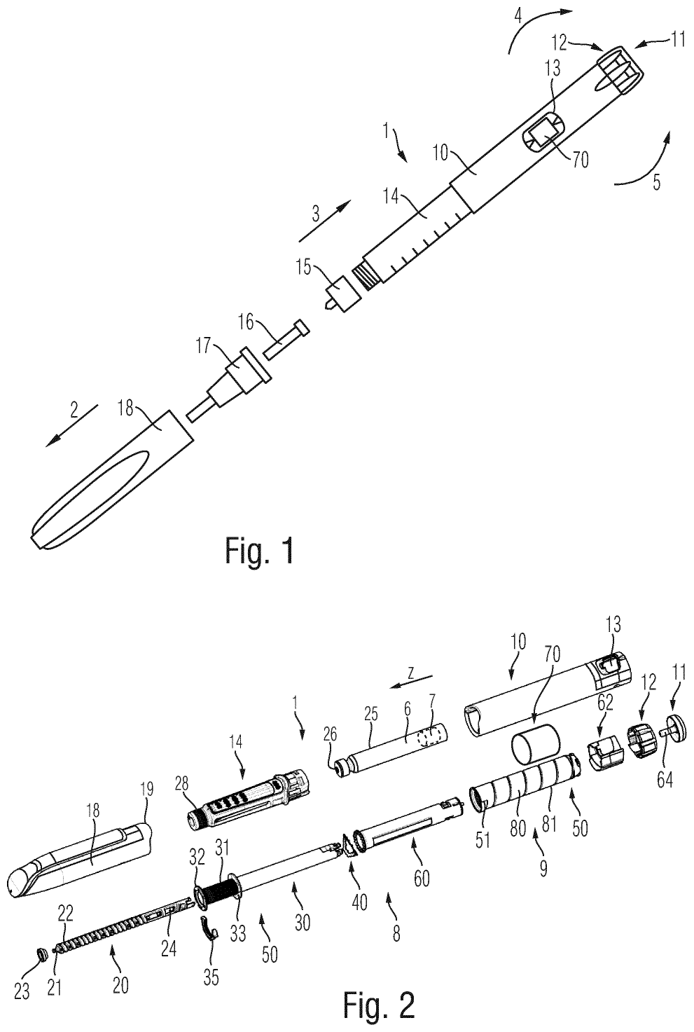

The injection device 1 as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 may comprise and form a main housing part configured to accommodate a drive mechanism 8 as shown in FIG. 2. The injection device 1 may further comprise a distal housing component denoted as cartridge holder 14. The cartridge holder 14 may be permanently or releasably connected to the main housing 10. The cartridge holder 14 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder 14 comprises a threaded socket 28 at its distal end to threadedly engage with a correspondingly threaded portion of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg). The dose dial 12 may comprise or may form a dose dial.

As shown further in FIGS. 1 and 2, the housing 10 comprises a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in display window 13 will be ejected from injection device 1. When the needle 15 of the injection device 1 remains for a certain time in the skin portion after the trigger 11 is pushed, a high percentage of the dose is actually injected into the patient's body. Ejection of an insulin dose may also cause a mechanical click sound, which is however different from the sounds produced when using the dose dial 12.

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

Furthermore, before using injection device 1 for the first time, it may be necessary to perform a so-called “prime shot” to remove air from the cartridge 6 and the needle 15, for instance by selecting two units of the medicament and pressing trigger 11 while holding the injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

The expelling or drive mechanism 8 as illustrated in more detail in FIG. 2 comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10. As will be explained below in greater detail, at least one of the stops is provided by a preselector stop feature 71 provided on a preselector 70.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement therebetween. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40 also acting as a clicker and the clutch 60 engaged, the drive sleeve 30, the spring or clicker 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 or provided on the preselector 70 to prevent further movement in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

The dose setting mechanism 9 as illustrated in FIG. 2 comprises at least the dose dial 12 and the number sleeve 80. As the dose dial 12 is rotated during and for setting of a dose the number sleeve 80 starts to rotate relative to the housing along a helical path as defined by the threaded engagement of its outer thread or helical groove 81 with a correspondingly shaped threaded section at the inside surface of the housing.

During dose setting and when the drive mechanism 8 or the dose setting mechanism 9 is in the dose setting mode the drive sleeve 30 rotates in unison with the dose dial 12 and with the number sleeve 80. The drive sleeve 30 is threadedly engaged with the piston rod 20, which during dose setting is stationary with regard to the housing 10. Accordingly, the drive sleeve 30 is subject to a screwing or helical motion during dose setting. The drive sleeve 30 starts to travel in proximal direction as the dose dial is rotated in a dose incrementing direction 4, e.g. in a clockwise direction. For adjusting of or correcting a size of a dose the dose dial 12 is rotatable in an opposite direction, hence in a dose decrementing direction 5, e.g. counterclockwise.

At least one of the drive sleeve 30 and the number sleeve 80 serves as a dose tracker 50 comprising a tracking stop feature 51. There is further provided a preselector 70 as a separate piece.

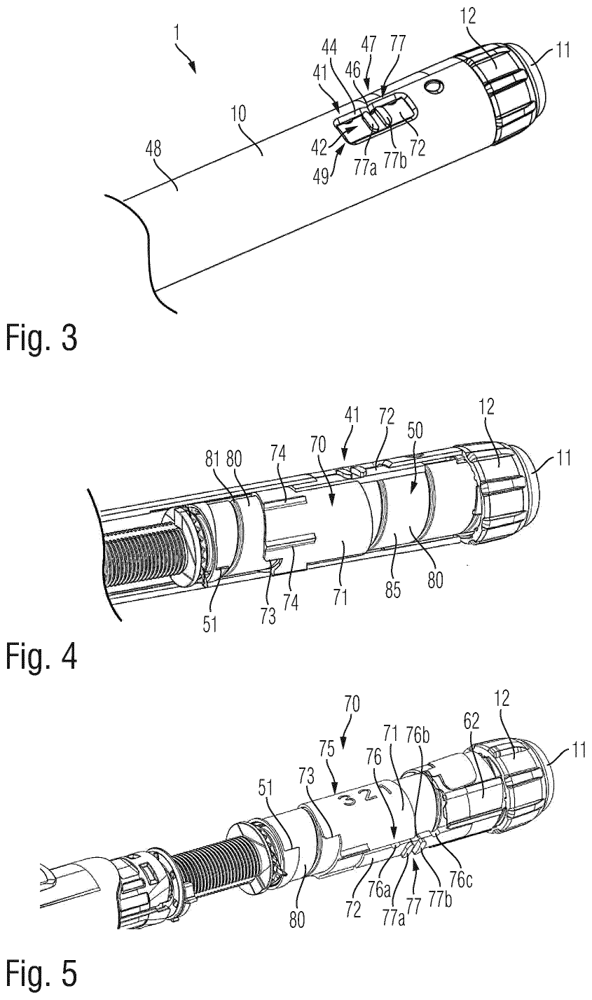

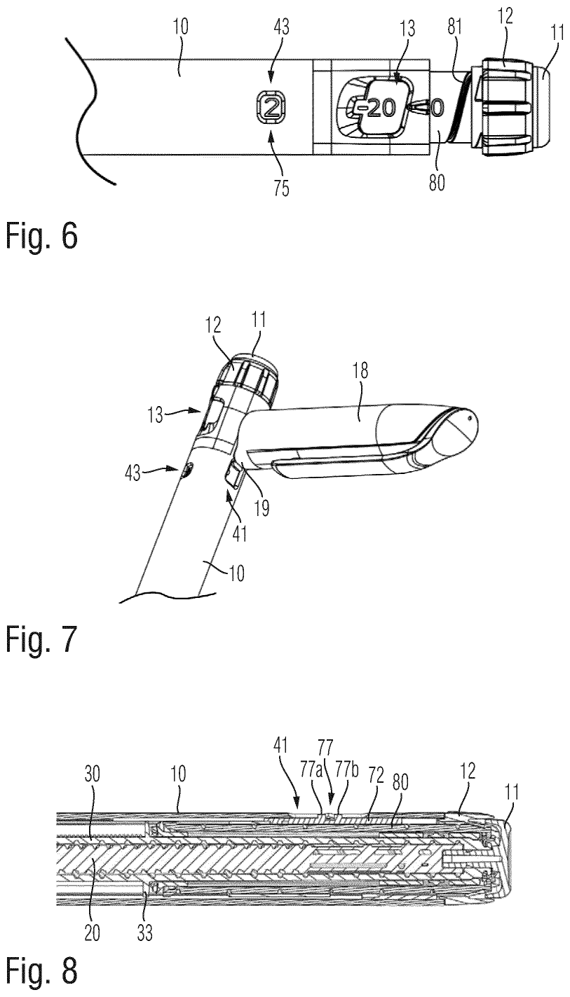

As illustrated with in FIGS. 3-8 the injection device, in particular the dose setting mechanism 9 thereof comprises a preselector 70 that is displaceable relative to the housing 10 of the injection device 1. The housing 10 comprises a tubular shaped sidewall 48. In the sidewall there is provided a recess 41. The recess is in form of a through opening 42. As illustrated in FIGS. 6 and 7 there is provided a dosage window 13 and a preselection window 43 in the sidewall 48 of the housing 10. The preselection window 43 and the dosage window 13 are separated from each other in longitudinal direction (z). In this way, information provided in the dosage window 13 and in the preselection window 43 can be acquired simultaneously.

The preselector 70 comprises a slider 72 that is slidably displaceable inside the recess 41 of the sidewall 48 of the housing 10. The slider 72 is longitudinally displaceable between a distal stop position and a proximal stop position. The slider 72 is a component of the preselector 70. The preselector 70 further comprises a sleeve portion 71. As illustrated in FIGS. 4 and 5 a distal end section of the sleeve portion 71 comprises a preselector stop feature 73. The preselector stop feature 73 is configured to engage with the tracking stop feature 51 provided at or near a distal end of the dose tracker 50. In the example as shown in FIGS. 3-8 the dose tracker 50 is formed by a number sleeve 80. The number sleeve 80 comprises a helical groove 81 that is threadedly engaged with a correspondingly shaped protrusion or with a male thread on an inside facing sidewall section of an insert piece 62 that is stationary attached and fixed to a proximal end of the sidewall 48 of the housing 10.

The preselector 70 is slidably displaceable along a displacement path 49 that extends along the sidewall 48 of the housing. In the presently illustrated examples the displacement path 49 extends parallel to the longitudinal axis (z) of the housing 10 of the injection device 1.

The preselector 70 is rotationally locked to the sidewall 48 of the housing. This is accomplished by at least one or several spline features 74 provided at an outside facing section of the sleeve portion 71 of the slider 70. The spline features 74 engage with correspondingly shaped grooves provided in the inside facing surface of the sidewall 48 of the housing 10. In this way, a sliding but non-rotational motion of the preselector 70 relative to the housing is provided.

As the dose dial 12 is rotated in a dose incrementing direction 4 the dose tracker 50 is rotated in the same direction and undergoes a proximally directed longitudinal displacement. The proximally directed displacement as well as the rotational displacement of the dose tracker 50 is abruptly stopped when the tracking stop feature 51 abuts with the preselector stop feature 73 of the preselector 70. The preselector 70 is fixable or lockable to the housing 10 and hence to the sidewall 48 at three different discrete longitudinal positions.

For this the preselector 70 is provided with a detent structure 76. As illustrated in FIG. 5, the detent structure 76 is provided at a side edge of the slider 72. Here, the detent structure 76 comprises three recesses 76a, 76b, 76c. The recesses 76a, b, c are configured to mechanically engage or to snap fit with a counter detent structure 46 provided at the sidewall 48 of the housing 10. The counter detent structure 46 is located at an edge 44 of the recess 41. The edge 44 confines the through opening 42 through the sidewall 48. The counter detent structure 46 may be located somewhat recessed from the outside surface of the sidewall 48. The counter detent structure comprises a radial protrusion to engage with one of the radial recesses 76a, 76b, 76c of the detent structure 76. In this way the preselector 70 is positionally fixed to the sidewall 48 of the housing. As illustrated further in FIGS. 4, 5 and 8 the slider 70 is radially sandwiched between an inside facing portion of the sidewall 48 and an outside facing portion of the number sleeve 80 or of the dose tracker 50.

The slider 72 and the sleeve portion 71 may be integrally formed. Hence, the preselector 70 may comprise only a single component. Compared to the injection device such as described in WO 2004/078239 A1, the present injection device only requires minor modifications. Here, only a single preselector 70 must be provided and the contour and shape of the sidewall 48 of the housing 10 requires a respective modification in order to slidably receive the preselector 70.

Apart from that no further modifications are required to a commercially distributed injection device that is configured for individual, variable and user selectable setting of doses of different size.

By displacing the preselector in longitudinal direction the axial or longitudinal position of the preselector stop feature 73 is varied accordingly. By moving or displacing the preselector 70 towards the proximal direction 3 compared to the illustration of FIG. 4 the dose tracker 50 and hence the number sleeve 80 is allowed to be rotated for one more entire revolution until the tracking stop feature 51 engages the preselector stop feature 73. Here the mutual engagement of the detent structure 76 and the counter detent structure 46 is adapted in conformity with the lead of the threaded engagement of the dose tracker 50 with the housing 10. The distance between the recesses 76a, 76b, 76c is determined by the axial displacement the dose tracker undergoes during a complete revolution with regard to the longitudinal axis. Hence, the detent structure 76 is a regular detent structure and the recesses 76a, 76b, 76c are equidistantly separated from each other.

The sleeve portion 71 of the preselector comprises a preselection indication 75. In the illustrated embodiment the preselection indication 75 comprises three consecutive numbers 1, 2, 3. Depending on the axial position of the preselector 70 relative to the housing 10 one of the preselection indications 75 will show up in the preselection window 43. As illustrated in FIG. δ the preselector 70 is in an intermediate position. Accordingly, number 2 is revealed in the preselection window 43. In the present example the preselection indication 75 is indicative of a maximum dose settable with the dose setting mechanism 9.

Here, the preselection indication 75 and the number as illustrated in the preselection window 43 has to be multiplied by 10 units. Accordingly, the preselection indication 75 as illustrated in FIG. δ indicates to a user of the device that a maximum size of 20 standard units of the medicament can be set. In FIG. 6, the maximum dose configuration is illustrated in which a dose indicating number 20 imprinted on the outside surface of the number sleeve 80 shows up in the dosage window 13.

As illustrated further in FIGS. 5 and 3 the slider 72 radially protrudes from the rather plane or even shaped outer surface of the sleeve portion 71. In this way the slider can be arranged inside the recess. The slider 72 may radially reach into the recess 41. The recess 41 may be formed as a recess portion on an inside surface of the sidewall 48. In this way the slider may experience a further sliding support. As illustrated further in FIGS. 3 and 5 the outside surface of the slider 72 is provided with a gripping structure 77. In the present example the gripping structure 77 comprises two radially outwardly protruding ribs 77a, 77b. The ribs 77a, 77b are separated by a predefined longitudinal distance. Between the ribs 77a, 77b there is provided a free space that is shaped to receive a protrusion 19 provided at a proximal end of the protective cap 18. Here, the protrusion 19 as provided on the protective cap can be used as a dedicated tool to enter the space between the radially outwardly protruding ribs 77a, 77b.

As indicated in FIG. 4, the gripping structure 77 and the ribs 77a, 77b thereof may be located radially recessed compared to the outside surface of the sidewall 48. They may be arranged flush with the outside surface of the sidewall so that they do not protrude from the sidewall. In this way an inadvertent movement or displacement of the preselector 70 can be effectively prevented. Displacement of the preselector 70 requires exertion of a well-defined force above a predefined force threshold that is governed by the mechanical interaction of the detent structure 76 and the counter detent structure 46. With the recessed or flushed location of the gripping structure 77 with regard to an outside surface of the sidewall 48 use of the protective cap 18 with its protrusion 19 or use of a further dedicated tool is required in order to move or to displace the preselector 70 from one preselection positional state to another preselection positional state.

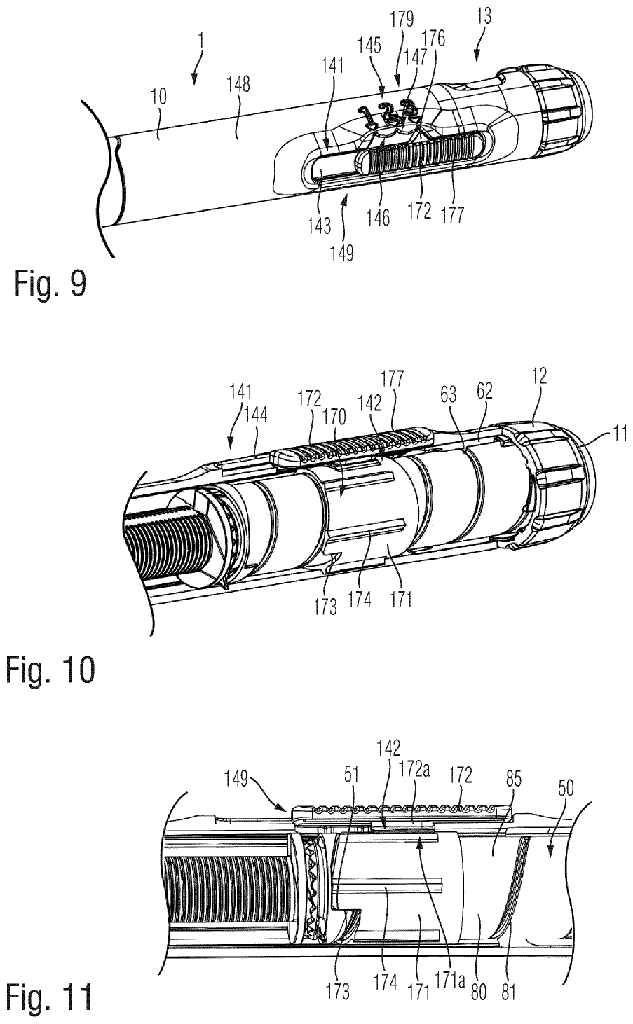

In FIGS. 9-10 a further example of a preselector 170 is shown. The general working principle of the preselector 170 and its interaction with the dose tracker 50 or with the number sleeve 80 is identical or substantially equivalent to the device and the preselector 70 as described in connection with FIGS. 3-8. Here, the housing 10 comprises a sidewall 148 with a recess 141. The recess 141 is of elongated shape and extends along the longitudinal axis of the housing 10. The recess 141 comprises a through opening 142 that extends as a longitudinal slit through a bottom section 143 of the recess 141. The recess 141 is circumferentially confined by a surrounding edge 144. The inner circumference of the edge 144 defines and/or coincides with the displacement path 149.

Inside the recessed portion formed by the bottom section 143 and the edge 44 the slider 172 is longitudinally displaceable between three distinct and discrete positions. For this the slider 172 comprises a detent structure 176 protruding radially from a main and substantially rectangular body of the slider 172. The detent structure 176 comprises a radial protrusion to mechanically engage with one of the correspondingly shaped recesses 147 of the counter detent structure 146 provided at the edge 144 of the recess 141. In the example as shown in FIG. 9 the protrusion of the detent structure 176 is of a tipped or triangular shape. It is located at a longitudinal central portion of the slider 172. The counter detent structure 146 is formed by a radial or tangential recess in the edge 144 of the recess 141.

Along the displacement path 149 there are provided three preselection indications 145 that are located at regular axial or longitudinal distances from each other. Each one of the preselection indications 145 denoted as 1, 2, 3 respectively, coincide with a recess 147 of the counter detent structure 146. The protrusion 176 acts as a pointer 179 pointing to one of the preselection indications 145 that aligns with the detent structure 176.

Apart from that the example as shown in FIGS. 9-11 is substantially identical to the example as described in connection with FIGS. 3-8. The contour and geometry of the sleeve portion 171 is substantially identical to the contour and the geometry of the sleeve portion 71 as described in connection with FIG. 4. Also here, the sleeve portion 171 comprises a preselector stop feature 173 to engage with the tracking stop feature 51. The sleeve portion 171 further comprises longitudinally extending spline features 174 to provide a rotational lock with the sidewall 148 of the housing 10.

Contrary to the example as described in connection with FIGS. 3-8 the preselector 170 is made of two pieces. Here, the slider 172 is provided as a piece and the sleeve portion 171 is provided as a separate piece. The sleeve portion 171 is assembled inside the housing 10. It is radially sandwiched between the dose tracker 50 and the sidewall 148 of the housing 10. Similar as described above the slider 172 is provided with a gripping structure 177 on its outside facing surface. The sleeve portion 171 comprises a connector 171a as indicated in FIG. 11. The connector 171a protrudes radially outwardly from the sleeve portion or comprises a recess in the outside facing surface of the sleeve portion 171. The slider 172 comprises a correspondingly shaped connector 172a that is configured to engage with the connector 171a. The two connectors 171a, 172a may form a snap fit or a positively interlocking connection.

The connector 172a of the slider 172 may comprise a radially inwardly extending protrusion or a recess to engage with the connector 171a. At least one of the two connectors 171a, 172a extends through a through opening 142 provided in the bottom section 143 of the sidewall 148 in the region of the recess 141. In this way, a mechanical engagement is provided between the sleeve portion 171 and the slider 172 that reaches radially through the sidewall 148 of the housing 10.

In FIGS. 10 and 11 it is further illustrated that the insert piece 62 comprises a radially inwardly extending protrusion 63 that is in engagement with the helical groove 81 or thread on the outside surface 85 of the number sleeve 80 and hence on the dose tracker 50.

The injection device 1 as illustrated in FIGS. 9-11 also comprises a dosage window 13 which is not explicitly illustrated. On the outside surface 85 of the number sleeve 80 or the dosing tracking member 50 there are provided various dose indicating numbers that show up in dosage window 13 when a dose of a respective size is set by rotating the dose dial 12, e.g. in a dose incrementing direction, such as clockwise.

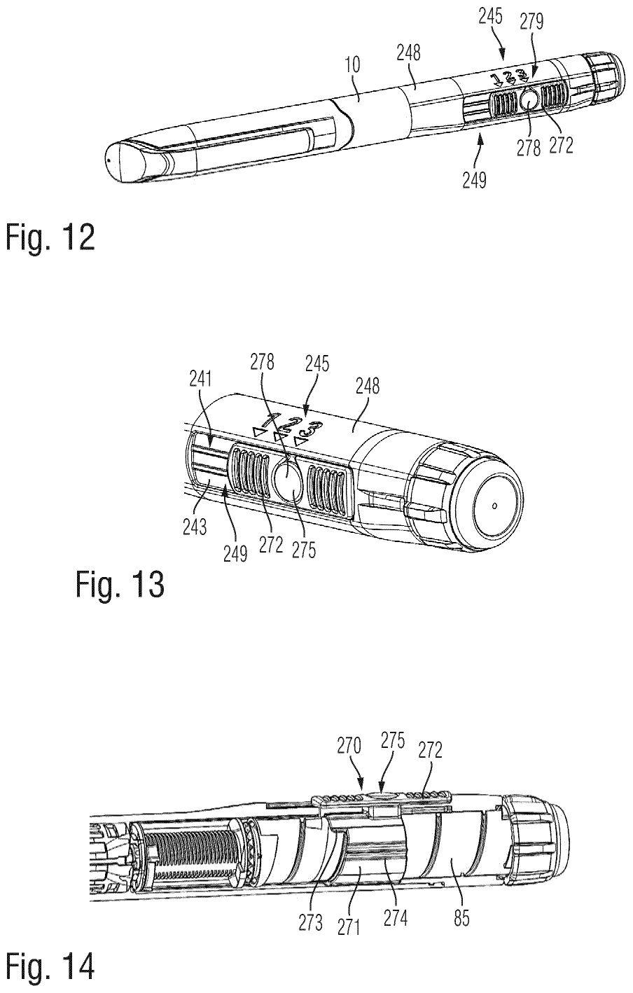

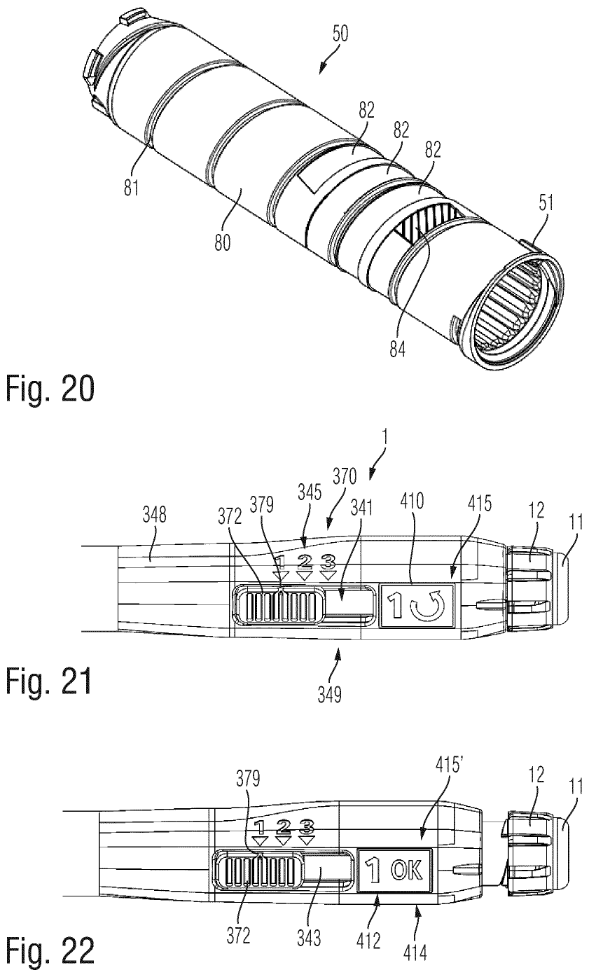

The general working principle of a further preselector 270 as shown with the example of FIGS. 12-20 is substantially equivalent and rather similar to the working principle of the preselector 170. The preselector 270 also comprises two parts, a sleeve portion 271 enclosing a longitudinal section of the dose tracker 50 and a slider 272 of elongated shape. Also here, the slider 272 is connectable to the sleeve portion 271 in the course of assembly of the injection device. After the sleeve portion 271 has been assembled inside the housing 10 the slider 272 can be connected therewith through a through opening 242 provided in a recess 241 of the sidewall 248 of the housing 10. Alternatively, the preselector 270 may be integrally formed and may comprise only a single piece component. Similar as described above the slider 272 is provided with a gripping structure 277 on its outside facing surface.

The recess 241 in the outside surface of the sidewall 248 comprises and forms a displacement path 249 along which the preselector 270 and hence the slider 272 thereof is longitudinally displaceable between three different discrete positions denoted as 1, 2, 3 in accordance to respective preselection indications 245 provided on the outside surface of the sidewall 248 as shown in FIG. 13.

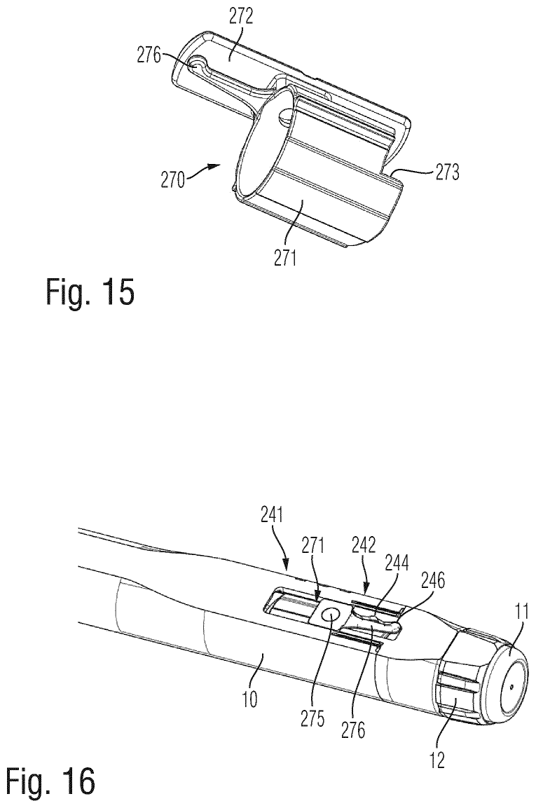

As shown in FIGS. 15 and 16 on a lower side of the slider 272 facing radially inwardly there is provided a tongue shaped detent structure 276 that is configured to mechanically engage with a correspondingly shaped counter detent structure 246 provided in a side edge 244 of the through opening 242 or of the recess 241 of the sidewall 248. Here, and contrary to the example as shown in FIG. 9 the mutual engagement between the slider 272 and the sidewall 248 is not visible from outside. The interaction between the detent structure 276 and the counter detent structure 246 is effectively covered by the slider 272. The slider 272 may be connected to the sleeve portion 271 from outside the housing 10.

Contrary to the examples as described above, the housing 10 and hence the sidewall 248 can be void of a dosage window 13. Instead, the slider 270 comprises an aperture 275 radially extending through the preselector 270 to reveal a portion of the outside surface 85 of the dose tracker 50 located underneath. In the present embodiment the aperture 275 is located in an overlapping portion of the slider 270 and the sleeve portion 271. Alternatively the aperture 275 may be exclusively provided in the slider 272.

Inside the aperture 275 there may be provided a magnifying lens 278. In this way the appearance of a dose indicating number located on the outside surface 85 of the dose tracker 50 or of the number sleeve 80 can be enlarged. This provides a better readability and discernability of numbers or dose indicating symbols on the outside surface 85 of the dose tracker 50. Alternatively or additionally the size of the aperture 275 as well as numbers or symbols provided on the outside surface 85 of the number sleeve 80 can be decreased thus enabling a further miniaturization of the injection device and of its dose setting mechanism 9.

The aperture 275 and hence the lens 278 may be provided in a longitudinal mid-section of the slider 272. The lens 278 may act and behave as a pointer 279. In the illustration of FIGS. 12 and 13 the lens 278 and hence the aperture 275 is tangentially or circumferentially aligned with the preselection indication 248 provided as number 3. Among three different selectable maximum dose sizes here the largest dose size has been exemplary selected.

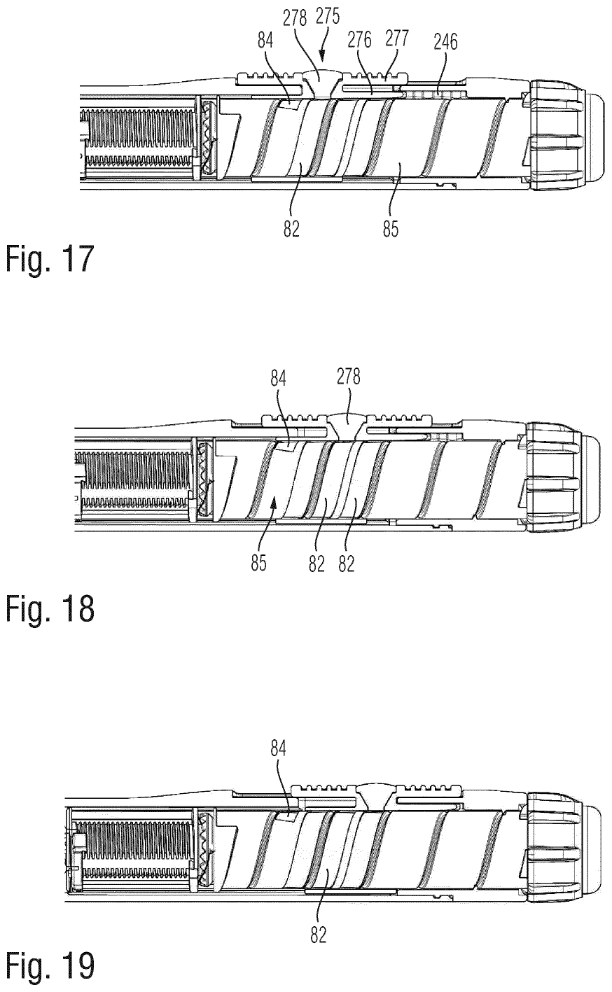

The outside surface 85 of the number sleeve 80 and hence of the dose tracker 50 comprises a first surface section 82 and a second surface section 84 as illustrated in FIGS. 17-20. In the present example the first surface section 82 has a visual appearance that differs from the visual appearance of the second surface section 84. First and second surface sections 82, 84 are non-overlapping. First and second surface sections 82, 84 extend between and/or along the helically shaped threaded structure on the outside surface 85 of the number sleeve 80 or of the dose tracker 50.

The second surface portion 84 is located near the distal end of the dose tracker 50 or number sleeve 80. It is located closer to the tracking stop feature 51 than the first surface section 82. In use and when selecting of a maximum allowable dose the second surface section 84 aligns with the aperture 275 when the tracking stop feature 51 of the dose tracker 50 engages with the preselector stop feature 273 of the sleeve portion 271. It is only upon reaching the predefined maximum dose size that the visual appearance of the aperture 275 changes. This is then a clear indication to the user, that the maximum allowable dose has been set. The change from the first surface section 82 overlapping with the aperture 275 towards the second surface section 84 overlapping with the aperture 275 is an indication and confirmation to the end user that a dose pre-selected by the preselection positional state of the slider 272 and hence of the preselector 270 has been reached. The device is then ready for dose dispensing.

Here it may of particular benefit, that the housing 10 can be void of a dosage window 13. General handling and operation of the device can be thus simplified. A user has only to select one of a number of available preselection positional states of the preselector. The user then has to rotate the dose dial 12 in the dose incrementing direction 4 until the visual appearance of the aperture 275 changes or until a predefined symbol appears in the aperture 275.

The assembly of the slider 272 and the sleeve portion 271 is slidably displaced inside or on the housing 10 and is prevented against rotation due to the splined engagement of the spline feature 274 on the outside surface of the sleeve portion 271 with a correspondingly shaped spline feature on the inside surface of the sidewall 248.

The further example of an injection device 1 as shown in FIGS. 21-25 is also void of a dosage window 13 in a sidewall 348 of the housing 10. Also here the preselector 370 comprises a sleeve portion 371 with a spline feature 374 for sliding engagement with the housing 10 and with a preselector stop feature 373 to engage with the tracking stop feature 51 of the dose tracker 50. The preselector 370 also comprises a slider 372 slidably displaceable relative to the housing 348 along a displacement path 349 that is defined and confined by a recess 341 in the outside surface of the housing 348. Laterally and hence along a side section of the recess 341 there are provided three preselection indications 345 that are provided with consecutive numbers 1, 2, 3, respectively.

In a way similar as described above in connection with the example according to FIGS. 12-20 the slider 372 is provided with a gripping structure 377 on its outside facing surface the slider 372 comprises a pointer 379 in a longitudinal mid-section. The pointer 379 aligns with one of the preselection indications 345, hence with any one of the numbers 1, 2 or 3. The present illustration of three discrete preselection indicators and three conceivable axial or longitudinal positions of the slider 372 is only exemplary. There are conceivable even less, e.g. at least two discrete positions or more discrete positions, such as 4, 5 or 6 discrete positions to which the slider 372 could be moved and in which the slider could be locked or fixed.

The slider 372 may be provided as a separate part to be connected to the sleeve portion 371 in the process of assembly of the injection device 1. The recess 341 also comprises a bottom section 343 that is intersected by a through opening 342 through which a mechanical connection between the slider 372 and the sleeve portion 371 extends.

The injection device 1 as illustrated in FIGS. 21-25 is void of a dosage window 13. For indicating a momentary state of the dose tracker 50 or of a number sleeve 80 the injection device 1 comprises a first position sensor 430 and a second position sensor 390. The first position sensor 430 is configured to determine or to detect a positional state of the dose tracker 50. The second position sensor 390 is configured to determine or to detect a positional state of the preselector 370. Moreover, the device may comprise a processor 420 to process signals obtainable from the second position sensor 390 and obtainable from the second position sensor 430, respectively.

The processor 420 may be configured to compare the positional state of the dose tracker 50. If the processor 420 determines that the positional state of the dose tracker 50 corresponds to the maximum dose positional state as governed and defined by the momentary preselection positional state of the preselector 370 the processor 420 is configured go provide a respective indicator on an electronic display 410. The second position sensor 390 is illustrated in FIG. 28.

The preselector 370 is equipped with a preselection indicator 375 located on an outside surface of the preselector 370. As illustrated in FIG. 25 the preselector 370 comprises a longitudinal extension 376 protruding proximally from a proximal end of the slider 372 and/or from a proximal end of the sleeve portion 371. Alternatively, the longitudinal extension 376 may also extend distally from the preselector 370. On its outside facing surface the extension 376 comprises the preselection indication 375. The preselection indication 375 could be one of a magnetically encoded or electrically conducting structure.

The preselection indication 375 may comprise an electric conductor. It may comprise a metal dome or a mental contact configured to connect or to bypass at least two electrical conductors connected to the processor 420. The preselection indication 375 is configured to interact with the position sensor 390 of the injection device 1. A position of the preselection indication 375 and hence of the slider 372 or of the preselector 370 is detectable by the second position sensor 390. For this the second position sensor 390 comprises three different sensor sections 391, 392, 393 as illustrated in FIG. 28. The sensor sections 391, 392, 393 are separated along the displacement path of the preselector 370. In the present embodiment the sensor section 391, 392, 393 are equidistantly spaced along the longitudinal axis (z).

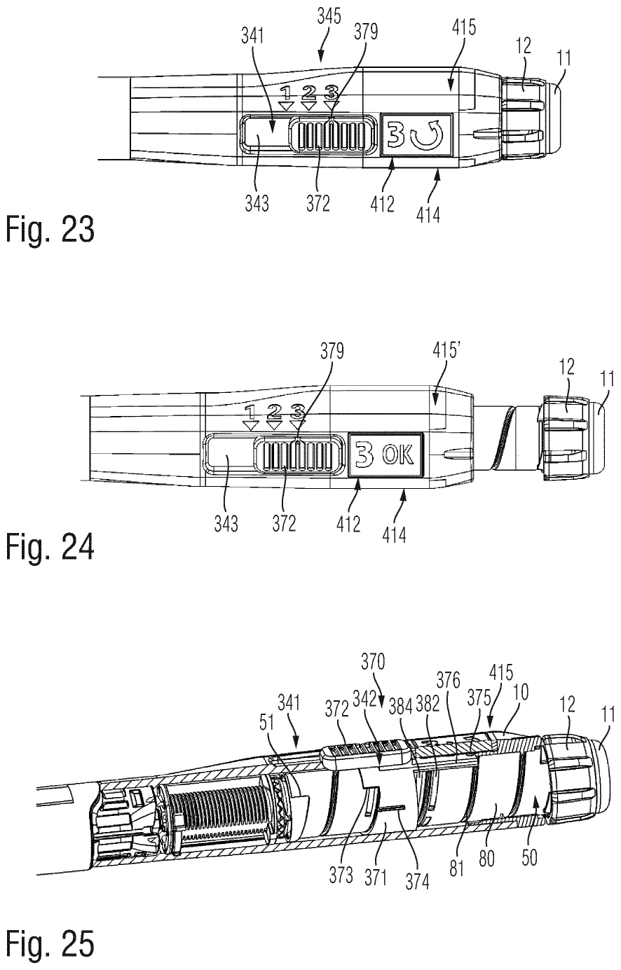

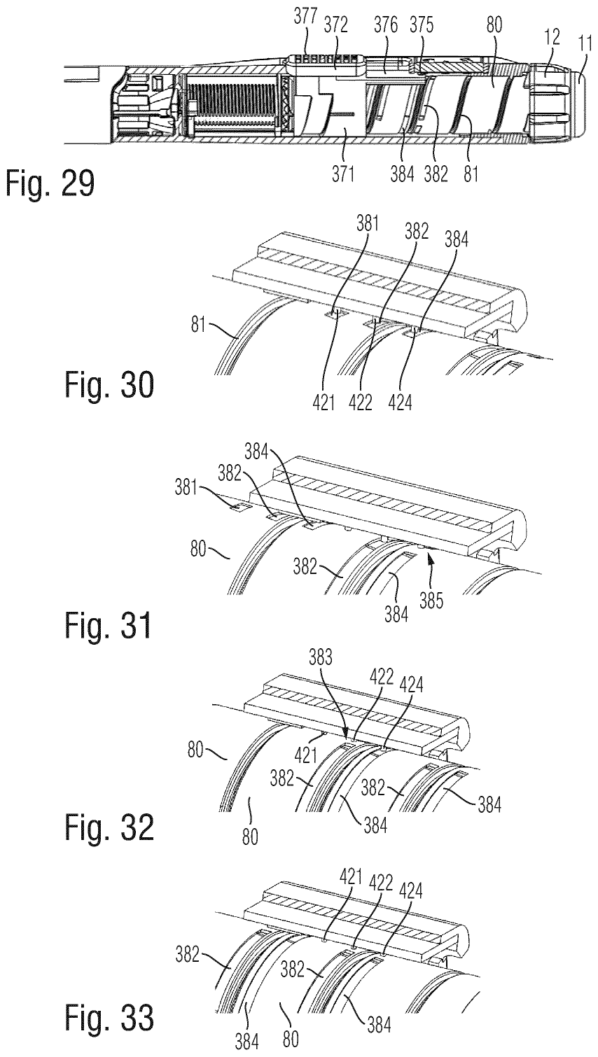

If the slider 372 and hence the preselector 370 is in a distal stop position as for instance illustrated in FIG. 21 the preselection indication 375 is axially aligned with the sensor section 393. Moving the slider 372 with its pointer 379 in alignment with the middle preselection indication 345 provided with number 2 the preselection indication 375 aligns and overlaps with the sensor section 392. When arriving in the proximal stop position as illustrated in FIGS. 23, 24, 29 and 27 the preselection indication 375 substantially overlaps or aligns with the sensor section 391. Between the preselection indication 375 and the sensor sections 391 there may be established an electrical contact. Depending on the specific implementation of the second position sensor 390 also a means for detecting a longitudinal or axial position of the preselection indication 375 could be implemented. The position detection may be based on one of an optical encoding and optical detector, a magnetic encoding and magnetic detection and/or an electric conductive connection between the preselection indicator 375 and one of the sensor sections 391, 392, 393.

The injection device 1 is provided with a display 410, configured as an electronic display. The display 410 comprises at least two display sections. In a first display section 412 a preselection indication in accordance with the actual position of the slider 372 is reproduced. As illustrated in FIGS. 21 and 22 the first display section 412 reproduces the number 1, being indicative for the smallest fixed dose to be set and dispensed with the present configuration of the dose setting mechanism 9. In the second display section 414 an indicator 415 is provided illustrating to the user whether the dose actually set coincides with the preselection indication or with the pre-selected dose.

In the configuration according to FIG. 21 the indicator 415 is indicative that the actually selected dose does not correspond to the preselection indication. The indicator 415 in FIG. 21 indicates to a user that dialing of the dose dial 12 is still necessary. If an end of dose configuration has been reached in which the tracking stop feature 51 engages with the preselector stop feature 373 the indicator 415 changes to an indicator 415′ as shown in FIG. 22 illustrating to the user that the pre-selected dose has been actually set.

FIGS. 23 and 24 are equivalent to FIGS. 21 and 22 with the exception that the preselector 370 has been positioned at a maximum dose configuration in which the pointer 379 is aligned with the preselection indication 345 that represents number 3.

The display 410 is typically provided with a processor 420 as shown in FIG. 28. The processor 420 is electrically connected to the second position sensor 390. Electrical signals generated by the position sensor are transmitted to the processor 420 in order to determine the longitudinal or axial position of the preselector 370 and hence of the slider 372.

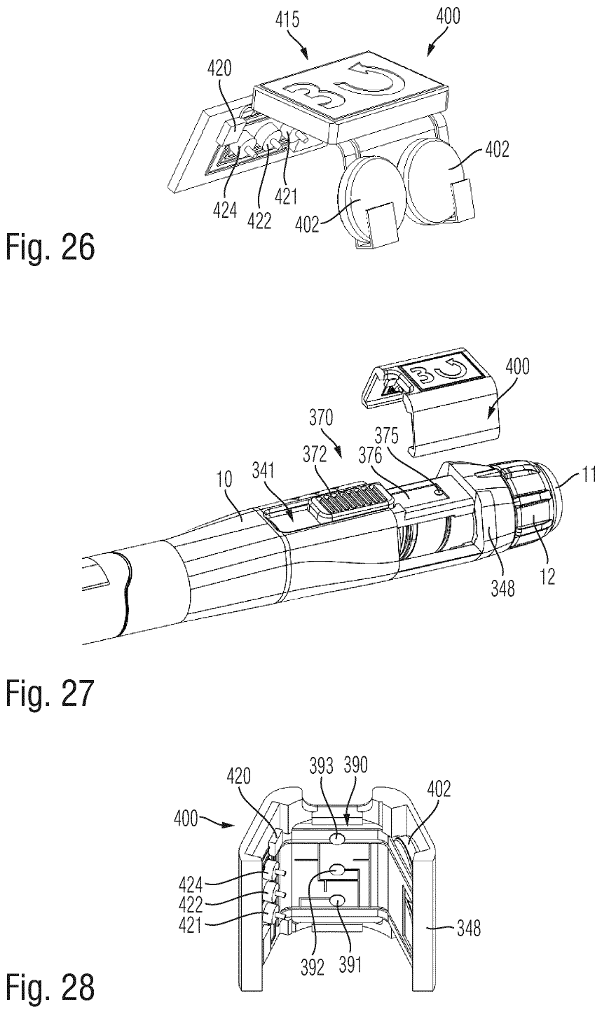

As illustrated in FIGS. 26 and 28 the processor 420 is further connected with the first position sensor 430 comprising a first detector element 422 and a second detector element 424. The first and the second detector elements 422, 424 are located and arranged at the inside of the sidewall 348 at a predetermined longitudinal distance. On the outside surface 85 of the number sleeve 80 or of the dose tracker 50 there are provided two different surface sections 382, 384. The first surface section 382 and the second surface section 384 extend along the helical groove 81.

The first and the second detector elements 422, 424 are stationary with the sidewall 348. Since the first and second surface sections 382, 384 extend at the same lead as the helical groove 81 and hence the threaded engagement between the dose tracker 50 and the sidewall 348 the first detector element 422 slides along the first surface section 382. The longitudinal or axial offset between the first and the second surface sections 382, 384 is identical to a longitudinal offset or separation between the first detector element 422 and the second detector element 424. As the number sleeve 80 or the dose tracker 50 rotates relative to the sidewall 348 the first and the second surface sections 382, 384 slide along the first and the second detector elements 422, 424, respectively.

In an initial configuration, hence in a zero dose configuration as for instance shown in FIG. 30, 23, 27 or 30 both detector elements 422, 424 are in direct contact or are coupled with a first and second surface section 382, 384, respectively. In the illustration of FIGS. 30-33 the distal direction points to the right whereas the proximal direction points to the left. The surface sections 382, 384 each comprise at least one interruption 383, 385 as illustrated in FIGS. 30 and 32. The first surface section 382 comprises an interruption 383 and the second surface section 384, hence the helical structure thereof comprises an interruption 385 as shown in FIG. 32. As one or both interruptions 383, 385 get in alignment with one or with both detector elements 422, 424 the interruption 383, 385 is detectable by the respective detector element 422, 424. In the configuration according to FIG. 30 both detector elements 422, 424 are in mechanical contact with the first and second surface section 382, 384. As the dose tracker 50 is rotated relative to the housing 10 and as a first maximum dose configuration as illustrated in FIG. 22 has been reached, the second detector element 424 is in alignment with the interrupt 385 while the first detector element 422 is still in contact with its respective first surface section 382 as shown in FIG. 31. This may represent a binary 1.

Rotating the number sleeve 80 or the dose tracker 50 further until it reaches a second maximum dose configuration in which the pointer 379 of the slider 372 aligns with the preselection indication 345 provided with number 2, the situation as illustrated in FIG. 32 arises, wherein the second detector element 424 is in contact with the second surface section 384 while the first detector element 422 is in alignment or in contact with an interrupt 383 of the first surface section 382. This configuration may represent a logical 2. In the configuration as shown in FIG. 33 that may correspond to the setting of FIG. 24 both detector elements 422, 424 are out of engagement or out of contact with regard to the first and second surface section 382, 384. This configuration may represent a logical 3.

The detector elements 422, 424 may be implemented as mechanical switches configured to generate an electrical signal as long as in contact with a respective surface section 382, 384.

When getting in mechanical contact with an interrupt 383, 385 the signal generated by the detector elements 422, 424 changes. Such a signal change is detectable and processable by the first position sensor 430 and hence by the processor 420 connected with the first position sensor 430. Since the processor 420 is further connected with the second position sensor 390 the three different situations as illustrated in FIGS. 31, 32 and 33 representing a logical 1, a logical 2 or logical 3 can be compared with the longitudinal or axial position of the preselection indication 375 that has been detected by means of the second position sensor 390.

The detector elements 421, 422, 424 can be implemented as mechanical switches when the surface sections 382, 384 and the interrupts 383, 385 comprise different radial height or depth on the outside surface 85 of the number sleeve 80 or of the dose tracker 50. For instance, the surface sections 382, 384 each comprise a longitudinal groove on the outside surface 85 of the number sleeve 80. The interrupts 383, 385 may flush with the outside surface of the number sleeve 80 or the dose tracker 50. When implemented as mechanical switches, the detector elements 422, 424 may each comprise a radially displaceable and spring biased pin sliding along the respective surface section 382, 384 as the number sleeve 80 is subject to a rotation relative to the housing 10. When a pin of one of the detector elements 422, 424 aligns with an interrupt 383, 385 the respective pin is depressed against the action of the spring. Such a depression is accompanied by a closing or opening of an electrical switch or contact inside the detector element 422, 424.

There is further illustrated an optional detector element 421 that serves as an on-off switch for the add-on device 400. The further detector element 421 is configured to engage with a further surface section 381 on the outside surface of the number sleeve 80. The surface sections 381 as illustrated in FIG. 31 comprises a confined recess in the outside surface of the number sleeve 80 or of the dose tracker 50. The recess and hence the surface section 381 is only slightly larger than the extent of the detector element 421. In an initial configuration or zero-dose configuration as for instance illustrated in FIG. 30 the detector element 421 is in alignment with the recess 381.

As soon as the number sleeve 80 is subject to a rotation the detector element 421 and the recess 381 get out of engagement. Consequently, the detector element 421 will be subject to a radially outwardly directed depression as it starts to slide as it starts to slide outside the recess 381 and hence along the outside surface of the number sleeve 80. In this way the add-on device 400 and hence the electronic components thereof, in particular its processor 420, is switched on and the status of the further detector elements 422, 424 can be monitored and processed. By means of a detector element 421 implemented as on/off switch, electric energy consumption of the add-on device 400 can be reduced and battery lifetime can be prolonged.

When after completion of a dose dispensing procedure the detector element 421 re-engages the recess 381 the add-on device 400 is switched off and electric energy can be saved.

Alternatively, the first and/or second surface sections 382, 384 can be electrically or magnetically encoded. For instance, the surface sections 382, 384 may be electrically conductive while the interrupts 383, 385 are electrically insulating or non-conductive. It is also conceivable that the surface sections 382, 384 and the interrupts 383, 385 mutually distinguish in terms of their visual appearance or light absorption characteristic as well and/or with regards to their magnetic properties. In this way also other encoding schemes based on an optic encoding or magnetic encoding can be generally implemented. With an optically or magnetically implemented encoding of the outside surface 85 of the dose tracker 50 or the number sleeve 80 also respective first and second detector elements 422, 424 should be implemented. Here, first and second detector elements 422, 424 may be implemented as light detectors or as magnetic sensors.

In a situation wherein the slider 372 is located near the proximal end of the displacement path 349 and wherein a maximum dose has been pre-selected, i.e. with the pointer 379 of the slider 372 aligns with the preselection indication 345 provided with number 3 the configurations as shown in FIGS. 30, 31 and 32 correspond to positions of the dose tracker 50 and hence of the number sleeve 80 that are smaller than the pre-selected dose size. Only when arriving in the proximal end position, in which the preselector stop feature 373 engages with the tracking stop feature 51 the electronic position detection of the dose tracker 50 coincides with the electronic position detection of the preselector 370. In such a situation the processor 420 is configured to switch the indicator 415 in the second display section 414 to confirm that the intended dose size has been set.

The display 410, the first position sensor 430, the second position sensor 390, the detector elements 422, 424 as well as the processor 420 can be permanently assembled inside the housing 10 of the injection device 1. These electronic components may belong to the injection device 1 and hence to the dose setting mechanism 9 thereof. The injection device 1 may be further equipped with one or several electric power sources 402, such as button batteries. The electric power sources 402 can be integrated into the housing 10 or can be detachably mounted inside the housing 10 and/or its sidewall 348. In a further embodiment and as indicated in FIG. 27 the electronic components as well as the electric power sources 402 can be all integrated into an add-on device 400 that may be detachably connectable to the housing 10, in particular to its sidewall. A detachable add-on device 400 may be usable with disposable injection devices that are equipped with a pre-filled cartridge 6 and which are intended to be discarded entirely after the content of the cartridge 6 has been used up.

Before discarding of a used injection device the add-on device 400 can be detached and can be attached to a new injection device 1. The add-on device 400 is further equipped with the above mentioned first and second position sensors 430, 390 in order to detect and to determine a position of the dose tracker 50 relative to the preselector 370.

List of reference numbers

| | |
|---|---|
| 1 | injection device |
| 2 | distal direction |
| 3 | proximal direction |
| 4 | dose incrementing direction |
| 5 | dose decrementing direction |
| 6 | cartridge |
| 7 | bung |
| 8 | drive mechanism |
| 9 | dose setting mechanism |
| 10 | housing |
| 11 | trigger |
| 12 | dose dial |
| 13 | dosage window |
| 14 | cartridge holder |
| 15 | injection needle |
| 16 | inner needle cap |
| 17 | outer needle cap |
| 18 | protective cap |
| 19 | protrusion |
| 20 | piston rod |
| 21 | bearing |
| 22 | first thread |
| 23 | pressure foot |
| 24 | second thread |
| 25 | barrel |
| 26 | seal |
| 28 | threaded socket |
| 30 | drive sleeve |
| 31 | threaded section |
| 32 | flange |
| 33 | flange |
| 35 | last dose limiter |
| 36 | shoulder |
| 40 | spring |
| 41 | recess |
| 42 | through opening |
| 43 | preselection window |
| 44 | edge |
| 46 | counter detent structure |
| 47 | protrusion |
| 48 | sidewall |
| 49 | displacement path |
| 50 | dose tracker |
| 51 | tracking stop feature |
| 60 | clutch |
| 62 | insert piece |
| 63 | protrusion |
| 64 | stem |
| 70 | preselector |
| 71 | sleeve portion |
| 72 | slider |
| 73 | preselector stop feature |
| 74 | spline feature |
| 75 | preselection indication |
| 76 | detent structure |
| 76a, b, c | recess |
| 77 | gripping structure |
| 77a, b | protrusion |
| 80 | number sleeve |
| 81 | groove |
| 82 | surface section |
| 84 | surface section |
| 85 | outside surface |
| 141 | recess |
| 142 | through opening |
| 143 | bottom section |
| 144 | edge |
| 145 | preselection indication |
| 146 | counter detent structure |
| 147 | recess |
| 148 | sidewall |
| 149 | displacement path |
| 170 | preselector |
| 171 | sleeve portion |
| 171a | connector |
| 172 | slider |
| 172a | connector |
| 173 | preselector stop feature |
| 174 | spline feature |
| 176 | detent structure |
| 177 | gripping structure |
| 179 | pointer |
| 241 | recess |
| 242 | through opening |
| 243 | bottom section |
| 244 | edge |
| 245 | preselection indication |
| 246 | counter detent structure |
| 249 | displacement path |
| 270 | preselector |
| 271 | sleeve portion |
| 272 | slider |
| 273 | preselector stop feature |
| 274 | spline feature |
| 275 | aperture |
| 276 | preselection indication |
| 277 | gripping structure |
| 278 | lens |
| 279 | pointer |
| 341 | recess |
| 342 | through opening |
| 343 | bottom section |
| 345 | preselection indication |
| 349 | displacement path |
| 370 | preselector |
| 371 | sleeve portion |
| 372 | slider |
| 373 | preselector stop feature |
| 374 | spline feature |
| 375 | preselection indication |
| 376 | extension |
| 377 | gripping structure |
| 379 | pointer |
| 381 | surface section |
| 382 | surface section |
| 383 | interrupt |
| 384 | surface section |
| 385 | interrupt |
| 390 | position sensor |
| 391 | sensor section |
| 392 | sensor section |
| 393 | sensor section |
| 400 | add-on device |
| 402 | electric power source |
| 410 | display |
| 412 | display section |
| 414 | display section |
| 415 | indicator |
| 420 | processor |
| 421 | detector element |
| 422 | detector element |
| 424 | detector element |
| 430 | position sensor |

1-15. (canceled)

16. An injection device for setting and injecting a dose of a medicament, the injection device comprising:
an elongated housing extending along a longitudinal axis and comprising a sidewall;
a dose tracker having a portion arranged inside the housing, the dose tracker comprising at least one tracking stop feature, wherein the dose tracker is at least one of translationally or rotationally displaceable relative to the housing during setting of a dose, wherein a positional state of the dose tracker relative to the housing is indicative of a size of the dose; and
a preselector comprising a preselector stop feature, wherein the tracking stop feature and the preselector stop feature are configured to mutually engage and to block a displacement of the dose tracker beyond a predefined maximum dose positional or rotational state,
wherein the preselector is at least one of translationally or rotationally displaceable relative to the housing along a displacement path between at least two preselection positional states, and wherein the preselector is lockable to the housing in any of the at least two preselection positional states.

17. The injection device according to claim 16, wherein the preselector is slidably displaceable along the displacement path extending along the sidewall of the elongated housing.

18. The injection device according to claim 16, wherein the preselector is rotationally locked to the elongated housing.

19. The injection device according to claim 16, wherein the preselector comprises a sleeve portion enclosing a longitudinal portion of the dose tracker.

20. The injection device according to claim 16, wherein the preselector comprises a slider slidably arranged in a recess in an outside facing portion of the sidewall.

21. The injection device according to claim 20, wherein the slider comprises a detent structure configured to engage with a correspondingly shaped counter detent structure of the sidewall.

22. The injection device according to claim 20, wherein one of the detent structure and the counter detent structure comprises a protrusion and wherein the other one of the detent structure and the counter detent structure comprises at least two recesses separated along the displacement path, and wherein any one of the at least two recesses is configured to receive the protrusion to lock the preselector against movement along the displacement path.

23. The injection device according to claim 22, wherein the protrusion is radial.

24. The injection device according to claim 16, wherein the preselector comprises a radially outwardly facing gripping structure comprising at least two radially outwardly protruding ribs separated from each other along the displacement path.

25. The injection device according to claim 24, wherein the gripping structure is radially recessed to an outside surface of the sidewall, wherein the gripping structure is substantially flush with the outside surface of the sidewall or protrudes radially outwardly from the outside surface of the sidewall.

26. The injection device according to claim 16, wherein the preselector comprises at least one preselection indicator on an outside facing surface portion of the preselector, and
wherein the elongated housing comprises a preselection window to reveal the preselection indicator.

27. The injection device according to claim 16, wherein the preselector comprises at least one preselection indicator on an outside surface portion of the preselector, and wherein the elongated housing comprises a position sensor configured to determine a position of the preselection indicator along the displacement path.

28. The injection device according to claim 16, wherein the elongated housing comprises at least two preselection indicators arranged along the displacement path, and wherein the preselector comprises a pointer coinciding with a position of at least one of the at least two preselection indicators.

29. The injection device according claim 16, wherein an outside surface of the dose tracker comprises a first surface section and a second surface section, wherein at least one of a haptic appearance, a visual appearance, a magnetic property or an electric property of the second surface section differs from a respective haptic appearance, a visual appearance, a magnetic property or an electric property of the first surface section.

30. The injection device according to claim 29, wherein the preselector comprises an aperture radially extending through the preselector to reveal a portion of the outside surface of the dose tracker.

31. The injection device according to claim 30, further comprising a slider, wherein the slider comprises a magnifying lens arranged in the aperture.

32. The injection device according to claim 16, further comprising a piston rod and a cartridge, wherein the cartridge comprises a barrel filled with the medicament and sealed in an axial proximal direction by a bung that is axially displaceable relative to the barrel by the piston rod.

33. The injection device according to claim 16, wherein the dose tracker is formed by a number sleeve that comprises a helical groove.

34. The injection device according to claim 33, wherein the helical groove is threadedly engaged with a correspondingly shaped protrusion or with a male thread on an insert piece that is attached to the sidewall of the elongated housing.

35. The injection device according to claim 34, wherein the protrusion or the male thread is on an inside facing sidewall of the insert piece, and wherein the insert piece is stationary fixed to a proximal end of the sidewall of the elongated housing.
